(19) United States
(12) Patent Application Publication
(10) Pub. No.: US 2015/0057602 A1
(43) Pub. Date: Feb. 26, 2015

(54) System and Method for Administering Peritoneal Dialysis

(71) Applicant: Roger Alan Mason, Dumas, AR (US)

(72) Inventor: Roger Alan Mason, Dumas, AR (US)

(21) Appl. No.: 14/468,598

(22) Filed: Aug. 26, 2014

Related U.S. Application Data
(60) Provisional application No. 61/869,915, filed on Aug. 26, 2013.

(51) Int. Cl. A61M 1/28

(52) U.S. Cl. CPC A61M 1/285; A61M 1/28
USPC 604/28; 604/29

(57) ABSTRACT

Systems and methods are provided for improved techniques associated with administering peritoneal dialysis. Embodiments of the invention relate to the continuous introduction and circulation of dialysate fluid in and through the peritoneal cavity. This constant influx of fresh fluid results in a perpetually high diffusion gradient between the toxin solute concentration of the blood and the dialysate fluid traversing the abdominal cavity, which promotes a much more efficient and rapid transfer of toxic solutes from the blood stream into the abdominal fluid. The fluid is continuously removed from the abdominal cavity and passed through an external filter using a pulsatile pump. The external filter cleanses the toxic solutes from the fluid before returning the fluid to the abdominal cavity. Embodiments of the invention also relate to improvements in catheters used to access the peritoneal cavity.

### CROSS REFERENCE OF RELATED APPLICATION

This application claims priority to U.S. Provisional Patent Application No. 61/869,915, filed Aug. 26, 2013.

### FIELD OF THE INVENTION

Embodiments of the invention relate generally to the field of treating a patient with dialysis.

### BACKGROUND

For a patient experiencing kidney failure, it eventually becomes necessary to replace kidney function with an alternate means of removing toxins that accumulate in the blood on a daily basis. Currently, there are two leading methods for achieving this objective: hemodialysis and peritoneal dialysis.

Hemodialysis refers to the process of removing blood from a patient using a needle in the patient's blood vessel via a plastic tubing. The blood is then circulated through an external filter before returning the purified blood to the patient via the plastic tubing and a second needle in the patient's blood vessel. This extremely onerous and time-consuming process is typically performed in a clinical setting, at least three times a week, and requires at least four hours of time per session.

The other principal procedure for removing toxins from the blood is peritoneal dialysis. This process is performed by placing one or two plastic catheters through the abdominal wall of a patient. Typically, the tip of the cavity remains in the abdominal cavity of the patient while the catheter chronically exits through the skin. The catheter is then used to place approximately two liters of pure, sterile fluid into the abdominal cavity. The peritoneum, or lining of the abdominal cavity, acts as a natural filter that encourages toxins to leave the patient's bloodstream, depositing them into the sterile fluid in the patient's abdomen. Traditionally, after toxins enter this fluid, the fluid is removed from the abdominal cavity and discarded.

Generally speaking, peritoneal dialysis is considered to be safer and less costly than hemodialysis. However, peritoneal dialysis is cumbersome and significantly restricts the activities of the patient. Indeed, peritoneal dialysis requires four to five cycles, lasting approximately three to four hours each, of dialysis per day. The patient must cope with a chronically extruding catheter which can be physically restrictive and cause discomfort, and can also be prone to infection. The (typically two liter) aliquots of fluid that are circulated through the abdomen must subsequently be discarded and replaced with fresh dialysate, which is costly as well as inefficient Still further, other disadvantages associated with peritoneal dialysis relate to the fact that the contact of the fluid with the abdominal lining for extended periods of time decreases the filtering capabilities of the peritoneum over time. So although peritoneal dialysis is actually the preferred method of dialysis for patients entering end stage kidney failure, it is only used by approximately 10% of patients requiring dialysis due to its inefficiency and the restrictiveness associated with patient activities.

Therefore, a solution is needed that remedies the current deficiencies and inefficiencies associated with peritoneal dialysis and allows dialysis to be accomplished in a more efficient and less costly manner. Those skilled in the art will appreciate that any such solution will need to address the problems associated with the chronically extruding catheter, as well as find a way to maintain a fresh pure dialysate fluid in the abdominal cavity while simultaneously finding a way to use the dialysate fluid efficiently.

### SUMMARY OF THE INVENTION

Embodiments of the invention are directed to a closed system for administering peritoneal dialysis to a patient, the system comprising a supply of dialysate, a first subcutaneous catheter and a second subcutaneous catheter both in communication with a peritoneal cavity of the patient, a first needle operable to access and connect the first subcutaneous catheter to a fluid pathway, a second needle operable to access and connect the second subcutaneous catheter to the fluid pathway, a pump operable to cause the supply of dialysate to flow through the peritoneal cavity, wherein the fluid travels into the peritoneal cavity via the first subcutaneous catheter and exits the peritoneal cavity via the second subcutaneous catheter, a filter for removing contaminants from the dialysate, wherein the pump causes the decontaminated dialysate to be recirculated through the fluid pathway and a container for storing contaminants removed by the filter from the dialysate.

Embodiments of the invention further include a method for administering peritoneal dialysis to a patient, the method comprising subcutaneously inserting a first subcutaneous catheter and a second subcutaneous catheter so that both the first subcutaneous catheter and the second subcutaneous catheter catheters are in communication with a peritoneal cavity of the patient, accessing the first subcutaneous catheter with a first needle and accessing the second subcutaneous catheter with a second needle, connecting the first subcutaneous catheter and the second subcutaneous catheter to a fluid pathway, pumping a supply of dialysate through the fluid pathway and into the peritoneal cavity, removing contaminants, after the dialysate has passed through the peritoneal cavity, from the dialysate, re-circulating the decontaminated dialysate through the fluid pathway and storing contaminants removed by the filter in a waste container.

### BRIEF DESCRIPTION OF THE DRAWINGS

The present invention is described in detail below with reference to the attached drawings:

FIG. 1 is a diagram illustrating current techniques associated with continuous ambulatory peritoneal dialysis;

FIG. 2 is a diagram illustrating a method for placing two catheters for dialysis in accordance with an embodiment of the invention;

FIG. 3 is a diagram illustrating the reduced volume fluid pathway in accordance with an embodiment of the invention; and

FIG. 4 is a diagram illustrating a method for placing two catheters for dialysis in accordance with an embodiment of the invention.

### DETAILED DESCRIPTION OF PREFERRED EMBODIMENTS

Embodiments of the invention relate to a technique for peritoneal dialysis that combines a unique catheter design with an alteration in typical dialysis techniques.

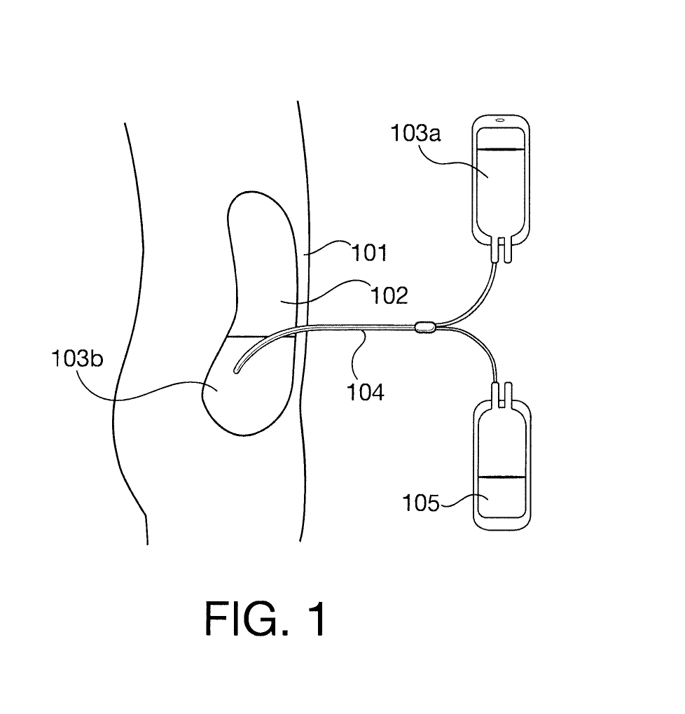

FIG. 1 is a diagram illustrating current techniques associated with continuous ambulatory peritoneal dialysis. These techniques operate by exchanging fluids and other dissolved substances (such as, e.g., urea, potassium, etc.) from the blood across the patient's peritoneum 101, which acts as a membrane, in the abdomen 102. Typically, systems and methods for peritoneal dialysis rely on surgical insertion of a catheter 104, prior to the commencement of dialysis treatment. After cleaning and any other required pre-treatment verification, a specified volume (commonly referred to as “dwell”) of fresh dialysate fluid 103a is introduced through catheter 104 in the abdomen 102 and flushed out during regular fluid exchanges throughout the day. The administered dialysate fluid 103b remains in the abdomen 102, while the peritoneum 101 acts as a natural filter that encourages toxins to leave the patient's bloodstream, depositing them into the (previously) administered dialysate fluid 103b in the patient's abdomen 102.

The efficiency of peritoneal dialysis depends on a diffusion gradient across the peritoneal membrane to drive the filtering process. The diffusion gradient is the presence of a greater concentration of particles in a solution on one side of a membrane or filter than on the other side of the membrane or filter. The difference in concentration drives the particles to transit from the fluid with the higher concentration of particles into the fluid with the lower concentration of particles.

Thus, during peritoneal dialysis, as the administered dialysate fluid 103b is introduced into the abdomen 102 and allowed to sit for three to four hours (or more), particles traverse from the higher concentration fluid (the bloodstream) into a low concentration fluid (the administered dialysate fluid 103b in the abdomen 102). As more particles enter the administered dialysate fluid 103b, the concentration difference between the fluid on both sides of the membrane of the peritoneum 101 decreases, and the speed of transfer of particles across the membrane slows. Eventually, the transit of particles becomes very slow, and the administered dialysate fluid 103b—now contaminated with toxins and other particles from the bloodstream—is removed and more fresh dialysate fluid 103a (without any particles) is introduced into the abdomen 102. The contaminated fluid is stored in waste fluid bags 105.

This cyclic process or “batch” process for peritoneal dialysis is cumbersome and restricts the activities of the patient significantly. It is also costly and inefficient, and may have to be repeated four to five times during the course of a twenty-four hour period, seven days a week.

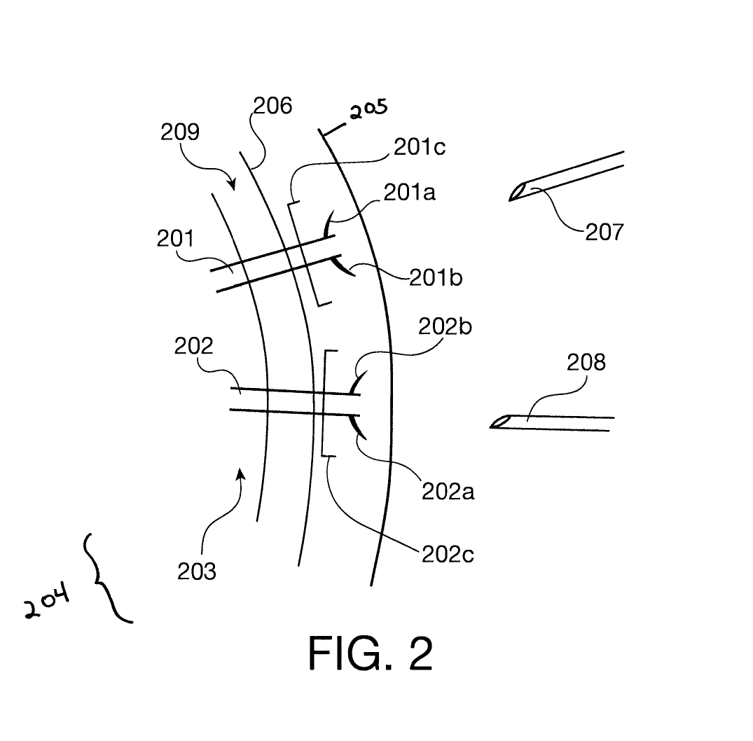

FIG. 2 is a diagram illustrating a method for placing two catheters for dialysis in accordance with an embodiment of the invention. In an embodiment of the invention, a first catheter 201 and a second catheter 202 are placed in communication with the abdominal cavity 203 of a patient 204. Those skilled in the art will appreciate that the catheters may be manufactured according to known methods from a variety of synthetic materials. For instance, in an embodiment of the invention, both catheters are made of plastic. The “access” ends of the first catheter 201 and second catheter 202 are buried beneath the skin 205 of patient 204 in the subcutaneous tissue 206. In embodiments of the invention, the dual catheters may also be combined into a single catheter comprising two ports and/or two arms.

In accordance with an embodiment of the invention, the first catheter 201 and second catheter 202, are “buried” and do not protrude chronically through the skin 205. Hence, the mobility of the patient 204 is increased, and activities that were not thought to be previously possible for peritoneal dialysis patients—such as swimming—are now possible. Still further, those skilled in the art will appreciate that this technique would be expected to decrease the incidence of catheter infection, improving the longevity of the catheter.

The first catheter 201 and second catheter 202 are accessed by placing needles 207 and 208 percutaneously through the skin 205 at the time of each dialysis session, into the “access” end of first catheter 201 and second catheter 202, respectively. In embodiments of the invention, first catheter 201 and second catheter 202 are designed with a small metal reservoirs 201a and 202a, respectively. First catheter 201 and second catheter 202 traverse the muscular abdominal wall 209 and end just below the skin 205, where metal reservoirs 201a and 202a reside, allowing fluid to be introduced into the abdominal cavity 203. The small metal reservoirs 201a and 202a may comprise a soft, synthetic membrane surface 201b and 202b, respectively, just below the skin 205 that is easily palpated and acts as a target for the needles 207 and 208, respectively. Additionally or alternatively, the first catheter 201 and second catheter 202 may also include a small skirt 201c and 202c, respectively. This skirt may surround the first catheter 201 and second catheter 202 just beyond their respective reservoirs, 201a and 202a, respectively, which will further decrease the incidence of infection that is often a problem for catheters chronically traversing the skin. In embodiments of the invention, the skirts may be made of polytetrafluoroethylene (i.e., Teflon®).

In an embodiment of the invention, the first catheter 201 and second catheter 202 are placed in the abdominal cavity 203 at “distant” sites to allow a steady introduction and removal of fluid from the abdominal cavity 203 at a constant, relatively high flow rate. Embodiments of the invention require needles 207 and 208, when percutaneously accessing the first catheter 201 and second catheter 202, respectively, to be secured at the time of each dialysis session in order to, for example, prevent leakage from the needle into the patient's abdominal cavity 203. This may be done by methods known in the art, such as, e.g., using clamps or locks. Some patients may also not require a buried catheter, and may find it preferable to use the embodiments of the invention described herein in conjunction with the use of a transcutaneous catheter. In embodiments of the invention, fluid may introduced to the abdominal cavity 203 via the first catheter 201, and removed via the second catheter 202.

Those skilled in the art will appreciate that embodiments of the invention in which there exists a continuous influx of pure dialysate fluid result in a perpetually high diffusion gradient between the toxin solute concentration of the blood and the pure dialysate traversing the abdominal cavity, promoting a significantly more efficient and rapid transfer of toxic solutes from the blood stream into the abdominal fluid.

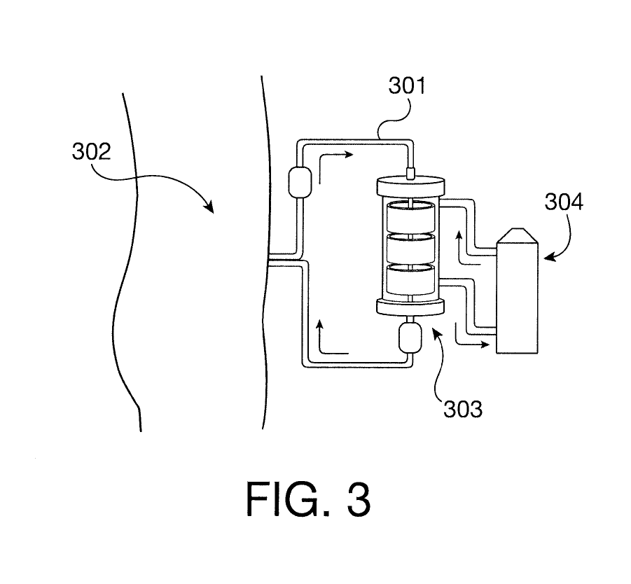

FIG. 3 is a diagram illustrating the reduced volume fluid circuit in accordance with an embodiment of the invention. In embodiments of the invention, the dialysate fluid 301 is continuously removed from the abdominal cavity 302 and passed through an external filter 303 using a pump 304. In embodiments of the invention, the pump 304 may be a pulsatile pump, peristaltic pump or any other type of pump known in the art. The external filter 303 cleanses any toxic solutes from the dialysate fluid 301, that may have been absorbed in abdominal cavity 302, before returning the dialysate fluid 301 to the abdominal cavity 302 so that the process can be repeated.

Those skilled in the art will appreciate that embodiments of the invention allow peritoneal dialysis to be conducted in much more efficient, less cumbersome, and less costly procedure. Further, because the dialysate fluid 301 is repeatedly cleansed as it recirculates through the external filter in the closed circuit, the systems and methods described herein will significantly reduce the cost of dialysis as only one bag of dialysate fluid is required (in contrast to the usual multiple bags of fluid currently required in peritoneal dialysis, which are discarded after the contaminated fluid is removed from the abdominal cavity). Still further, the decreased volume of dialysate will correspond with a lower lifetime exposure of the peritoneal membrane to dialysate. Although the dialysate fluid 301 may refer to fluid compositions that are well known in the art, those skilled in the art will appreciate that certain compositions may also exist that optimize the embodiments of the invention detailed and described herein.

It is also contemplated that increasing blood flow to the peritoneum in accordance with embodiments of the invention may increase the effectiveness of the filtration process.

Those skilled in the art will appreciate that this may be accomplished by administering a particular substance to the patient via the dialysate.

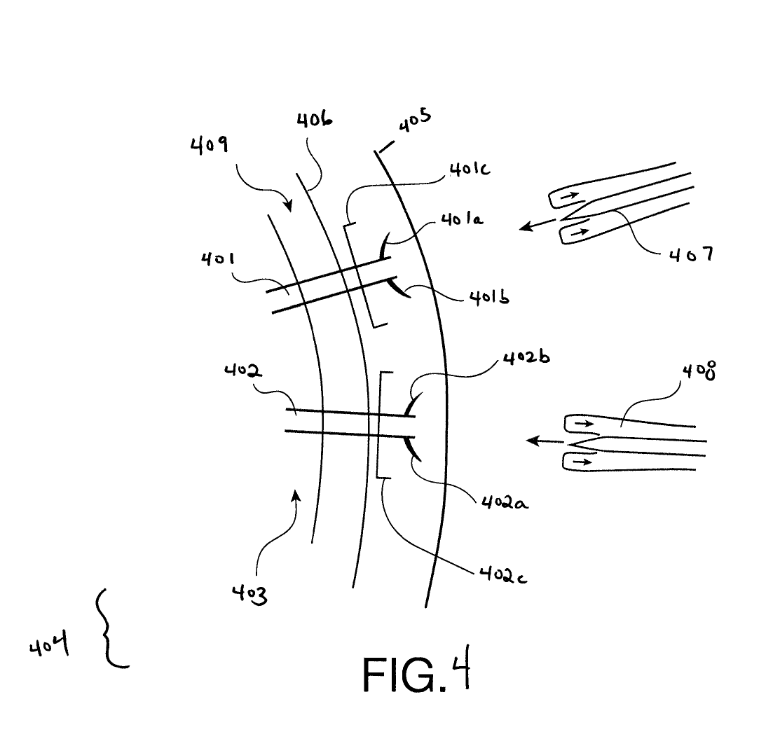

FIG. 4 is a diagram illustrating a method for placing two catheters for dialysis in accordance with an embodiment of the invention. In an embodiment of the invention, a first catheter 401 and a second catheter 402 are placed in communication with the abdominal cavity 403 of a patient 404. The “access” ends of the first catheter 401 and second catheter 402 are buried beneath the skin 405 of patient 404 in the subcutaneous tissue 406.

As shown in FIG. 4, first catheter 401 and second catheter 402 are accessed by placing needles 407 and 408 percutaneously through the skin 405 at the time of each dialysis session, into the “access” end of first catheter 401 and second catheter 402, respectively. Needles 407 and 408 may include various features such as, e.g., a retractable needle guide of the type depicted in FIG. 4 or any other type known in the art. In an embodiment of the invention, needles 407 and 408 would not be exposed until the guide retracts upon contact with skin 405. Those skilled in the art will appreciate that a compatible distal locking mechanism may also be used to secure needles 407 and 408 in place when they protrude. Among other benefits, a retractable guide may also improve safety and decrease adverse incidents associated with the administration of the process.

Accordingly, in an embodiment of the invention first catheter 401 and second catheter 402 are designed with a small metal reservoirs 401a and 402a, respectively. First catheter 401 and second catheter 402 traverse the muscular abdominal wall 409 and end just below the skin 405, where metal reservoirs 401a and 402a reside, allowing fluid to be introduced into the abdominal cavity 403. The small metal reservoirs 401a and 402a may comprise a soft, synthetic membrane surface 401b and 402b, respectively, just below the skin 405 that is easily palpated and acts as a target for the needles 407 and 408, respectively. First catheter 401 and second catheter 402 may also include small skirts 401c and 402c, respectively, which may surround the first catheter 401 and second catheter 402 just beyond their respective reservoirs, 401a and 402a, respectively. This may further decrease the incidence of infection that is often a problem for catheters chronically traversing the skin.

It will be appreciated by those skilled in the art that the various embodiments and features of the presently disclosed invention may be used in any combination, as the combination of these embodiments and features are well within the scope of the invention. While the foregoing description includes many details and specificities, it is to be understood that these have been included for purposes of explanation only, and are not to be interpreted as limitations of the present invention. It will be apparent to those skilled in the art that other modifications to the embodiments described above can be made without departing from the spirit and scope of the invention. Accordingly, such modifications are considered within the scope of the invention as intended to be encompassed by the following claims and their legal equivalents.

While particular embodiments of the invention have been illustrated and described in detail herein, it should be understood that various changes and modifications might be made to the invention without departing from the scope and intent of the invention. From the foregoing it will be seen that this invention is one well adapted to attain all the ends and objects set forth above, together with other advantages, which are obvious and inherent to the systems and methods. It will be understood that certain features and sub-combinations are of utility and may be employed without reference to other features and sub-combinations.

What is claimed is:

1. A closed system for administering peritoneal dialysis to a patient, the system comprising:
a supply of dialysate;
a first subcutaneous catheter and a second subcutaneous catheter both in communication with a peritoneal cavity of the patient;
a first needle operable to access and connect the first subcutaneous catheter to a fluid pathway;
a second needle operable to access and connect the second subcutaneous catheter to the fluid pathway;
a pump operable to cause the supply of dialysate to continuously flow through the peritoneal cavity, wherein the fluid travels into the peritoneal cavity via the first subcutaneous catheter and exits the peritoneal cavity via the second subcutaneous catheter;
a filter for removing contaminants from the dialysate, wherein the pump causes the decontaminated dialysate to be recirculated through the fluid pathway; and
a container for storing contaminants removed by the filter from the dialysate.

2. The system of claim 1, wherein the pump is a peristaltic pump.

3. The system of claim 1, wherein at least one of the first subcutaneous catheter and the second subcutaneous catheter comprises a small metal reservoir.

4. The system of claim 3, wherein the surface of the small metal reservoir comprises a soft, synthetic membrane.

5. The system of claim 1, wherein:
both the first subcutaneous catheter and the second subcutaneous catheter comprise a small metal reservoir; the first subcutaneous catheter is surrounded by a first skirt; and
the second subcutaneous catheter is surrounded by a second skirt.

6. The system of claim 5, wherein the skirt surrounding each catheter is closer to the peritoneal cavity than the reservoir.

7. The system of claim 5, wherein the first skirt and second skirt are made of polytetrafluoroethylene.

8. The system of claim 1, wherein the supply of dialysate is limited to a single bag of dialysate fluid.

9. The system of claim 8, wherein the single bag of dialysate fluid contains between 2.0 and 2.5 liters of dialysate fluid.

10. A method for administering peritoneal dialysis to a patient, the method comprising:
subcutaneously inserting a first catheter and a second catheter so that both the first catheter and the second catheter are in communication with a peritoneal cavity of the patient;
accessing the first catheter with a first needle and accessing the second catheter with a second needle;
connecting the first catheter and the second catheter to a closed fluid pathway;
pumping dialysate fluid through the closed fluid pathway and into the peritoneal cavity;
removing contaminants, after the dialysate fluid has passed through the peritoneal cavity, from the dialysate fluid;
re-circulating the decontaminated dialysate fluid through the closed fluid pathway so that the dialysate fluid is constantly being circulated through the closed fluid pathway; and
storing contaminants removed by the filter in a waste container.

11. The method of claim 10, wherein the dialysate fluid is pumped through the closed fluid pathway using a peristaltic pump.

12. The method of claim 10, wherein at least one of the first catheter and the second catheter comprises a small metal reservoir.

13. The method of claim 12, wherein the surface of the small metal reservoir comprises a soft, synthetic membrane.

14. The method of claim 10, wherein the dialysate fluid is limited to a single bag of dialysate fluid.

15. The method of claim 14, wherein the single bag of dialysate fluid contains between 2.0 and 2.5 liters of dialysate fluid.
